(19) United States
(12) Patent Application Publication
(10) Pub. No.: US 2026/0151113 A1
(43) Pub. Date: Jun. 4, 2026

(54) USER-SPECIFIC ADAPTIVE SEGMENTATION IN MEDICAL IMAGING

(71) Applicant: GE Precision Healthcare LLC, Waukesha, WI (US)

(72) Inventors: Edina Timkó, Budapest (HU); Anuprriya Gogna, Bengaluru (IN)

(73) Assignee: GE Precision Healthcare LLC, Waukesha, WI (US)

(21) Appl. No.: 18/969,066

(22) Filed: Dec. 4, 2024

(51) Int. Cl. A61B 8/00; G06T 7/10

(52) U.S. Cl. CPC A61B 8/5215; A61B 8/465; G06T 7/10; G06T 2207/10132; G06T 2207/20081

(57) ABSTRACT

Systems and methods are provided for providing adaptive and intelligent segmentation processes that support personalization during ultrasound scanning. In one example, an ultrasound imaging system includes a processing circuit having a processor coupled to a memory device storing instructions thereon that, when executed, cause the processing circuit to perform operations including generating an ultrasound image based on image data obtained by a transducer; segmenting an anatomical structure in the ultrasound image using a segmentation algorithm; presenting, on a display of the ultrasound imaging system, the ultrasound image including the segmentation of the anatomical structure; receiving, via the display, an input from a user, the input including an adjustment to the segmentation of the anatomical structure; adjusting the segmentation of the anatomical structure based on the input; presenting, via the display, the ultrasound image with the adjusted segmentation of the anatomical structure; and updating the segmentation algorithm based on the input.

### FIELD

Embodiments of the subject matter disclosed herein relate to ultrasound imaging, and more particularly, to providing adaptive and intelligent segmentation of an anatomical region during an ultrasound imaging workflow based on personalized preferences of a user performing the ultrasound imaging workflow.

### BACKGROUND

Medical images obtained during a medical imaging procedure depict various anatomical features and structures. In some instances, such anatomical features and structures are automatically segmented in the medical images to assist a viewer (e.g., a technician, a doctor, etc.) when analyzing the medical images.

### SUMMARY

An embodiment relates to an ultrasound imaging system. The ultrasound imaging system includes a transducer configured to transmit and receive an ultrasound signal, a matching layer configured to have an acoustic impedance between a tissue to be imaged and a material of the transducer, a damping block configured to absorb ultrasound energy, and a processing circuit. The processing circuit includes a processor coupled to a memory device storing instructions thereon that, when executed, cause the processing circuit to perform operations including generating an ultrasound image based on image data obtained by the transducer. The operations include segmenting an anatomical structure in the ultrasound image using a segmentation algorithm. The operations include presenting, on a display of the ultrasound imaging system, the ultrasound image including the segmentation of the anatomical structure. The operations include receiving, via the display, an input from a user, where the input includes an adjustment to the segmentation of the anatomical structure. The operations include adjusting the segmentation of the anatomical structure based on the input. The operations include presenting, via the display, the ultrasound image with the adjusted segmentation of the anatomical structure. The operations include updating the segmentation algorithm based on the input.

Another embodiment relates to medical imaging system including a processing circuit having a processor coupled to a memory device storing instructions thereon that, when executed, cause the processing circuit to perform operations. The operations include generating a medical image. The operations include segmenting an anatomical structure in the medical image using a segmentation algorithm. The operations include presenting, on a display of the medical imaging system, the medical image including the segmentation of the anatomical structure. The operations include receiving, via the display, an input from a user, where the input includes an adjustment to the segmentation of the anatomical structure. The operations include adjusting the segmentation of the anatomical structure based on the input. The operations include presenting, via the display, the medical image with the adjusted segmentation of the anatomical structure. The operations include updating the segmentation algorithm based on the input from the user.

Another embodiment relates to a method. The method includes generating, by a processing circuit of a medical imaging system, a medical image. The method includes segmenting, by the processing circuit, an anatomical structure in the medical image using a segmentation algorithm. The method includes presenting, by the processing circuit, the medical image including the segmentation of the anatomical structure. The method includes receiving, by the processing circuit, an input from a user, where the input includes an adjustment to the segmentation of the anatomical structure. The method includes adjusting, by the processing circuit, the segmentation of the anatomical structure based on the input. The method includes presenting, by the processing circuit, the medical image with the adjusted segmentation of the anatomical structure. The method includes updating the segmentation algorithm based on the input from the user.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of a processing circuit used in the ultrasound imaging system of FIG. 1, according to an example embodiment.

FIG. 3 is a flow chart illustrating a method for providing adaptive segmentation using the ultrasound imaging system of FIG. 1, according to an example embodiment.

FIG. 4 is a flow chart illustrating a method for updating the segmentation provided during the method of FIG. 3 according to user-preferences, according to an example embodiment.

FIG. 5 is a block diagram of a workflow for performing user-specific adaptive segmentation using the ultrasound imaging system of FIG. 1, according to an example embodiment.

FIG. 6A is an illustration of an ultrasound image including user-defined adjustments to a default segmentation of an anatomical feature, according to an example embodiment.

FIG. 6B is an illustration of the ultrasound image of FIG. 6A including an adjusted segmentation based on the user-defined adjustments, according to an example embodiment.

FIG. 7A is an illustration of an ultrasound image including default segmentations of a plurality of anatomical features, and user-defined adjustments to one of the default segmentations, according to an example embodiment.

FIG. 7B is an illustration of the ultrasound image of FIG. 7A including adjusted segmentations of the plurality of anatomical features based on the user-defined adjustments to the one of the default segmentations, according to an example embodiment.

FIG. 8 is an illustration of a user interface configured to facilitate user-specific adaptive segmentation, according to an example embodiment.

### DETAILED DESCRIPTION

Referring generally to the figures, systems and methods for providing user-specific adaptive segmentation of anatomical structures in medical images are disclosed. The systems and methods disclosed herein adjust segmentations based on user input and store received user input such that segmentations automatically generated during successive medical imaging procedures more closely align with user-preferences and behavior.

During an ultrasound, contouring and/or measurements of anatomical structures in ultrasound images are often subject to considerable inter-observer variability. Such variability primarily arises out of image quality variations due to patient characteristics and acquisition settings. Further, expected segmentation outputs may vary depending on user-, site-, patient-, and/or demographic-specific clinical protocols. However, conventional segmentation algorithms used to perform automatic detection of anatomical structures are influenced by the quality and distribution of underlying data, and thus cannot fully accommodate user- and/or site-specific preferences or examination protocols. Therefore, automatically generated segmentations of anatomical structures often require subsequent user edits in order to accommodate for the user- and/or site-specific preferences or examination protocols. For example, such edits may include manual editing of the ultrasound image or a restart of the entire process in order to generate a user preferred result (e.g., by applying new image acquisition settings, etc.) Thus, existing systems for automatically generating segmentation of anatomical structures in ultrasound images ultimately decrease workflow efficiency and increase examination time.

To bridge the gap between providing a generic solution to generating anatomical segmentations and providing intra-observer variability (e.g., personalization), the systems and methods disclosed herein provide an adaptive algorithm/workflow to contour (e.g., segment) and/or measure any 2D or 3D structure in a medical image. Furthermore, the adaptive workflow described herein increases efficiency regarding user-provided adjustments to anatomical segmentations. In addition, the workflow adapts to user preference by learning user behavior and prompts in order to present improved automated segmentation results over time.

This disclosure relates to a novel, intelligent, and universally applicable system for adjustment of default/automated segmentations based on a received user prompt for any 2D or 3D homogenous structure or anatomy in a medical image. The systems and methods described herein improve workflow efficiency and decrease examination time without compromising accuracy for a wide range of medical imaging applications. The adaptive workflow described herein is also configured to update the underlying algorithm over time in order to adapt to user preference and ensure more appropriate segmentation results in successive medical imaging procedures.

The implementations described herein address a technical problem by providing enhanced data integration and analysis capabilities, which deliver a particular technical solution that streamlines and refines generation and transmittal of ultrasound images. The systems described herein are implemented to improve how user input is synthesized and utilized from various ultrasound scans to provide user-specific adaptive segmentation of anatomical structures in an ultrasound image. By integrating data related to a specific user, these systems provide real-time, intelligent segmentation of anatomical features and structures during an ultrasound scan. Accordingly, this approach provides a specific technical improvement to various technical problems, including those set forth herein.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

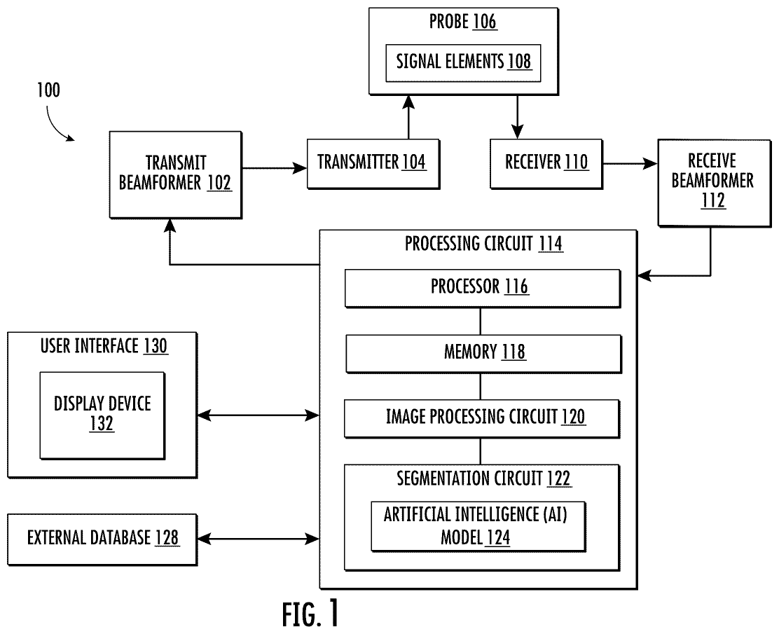

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient. Although the systems and methods are described herein in the context of the ultrasound imaging system 100, it should be appreciated that the user-specific adaptive segmentation may be performed using any of a variety of medical imaging systems (e.g., medical resonance imaging, x-ray, computed tomography, positron emission tomography, etc.).

Examples of a procedure performed using the ultrasound imaging system 100 may include a second trimester fetal examination, a pelvic examination, fibroid and follicle monitoring, and so on. In each of these examples, two-dimensional (2D) and/or three-dimensional (3D) contouring of relevant anatomical structures and/or measurements (e.g., caliper placement) are an integral part of the procedure. Taking the second trimester fetal examination as a specific instance, the contouring is critical to measuring biparietal diameter, head circumference, abdominal circumference, etc. Similarly, contouring impacts area and diameter measurements taken during fibroid and follicle monitoring. Therefore, operators of the ultrasound imaging system 100 rely on the contour in the ultrasound images generated therefrom to assess fetal health, monitor growth of anatomical structures, and perform other medical assessments.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data and 2D color flow data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a left atrial appendage (LAA) at a first location of the probe 106. Then, the position sensor may track the movement of the probe 106 relative to the LAA in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during a fetal examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, and a segmentation circuit 122. In this way, the processing circuit 114 may be structured or configured to execute or implement the instructions, commands, and/or control processes described herein with respect to the processor 116, the memory 118, the image processing circuit 120, and the segmentation circuit 122. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.).

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

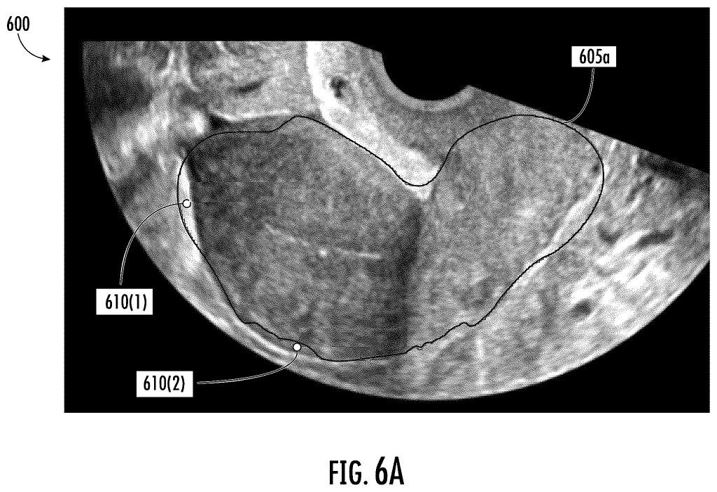

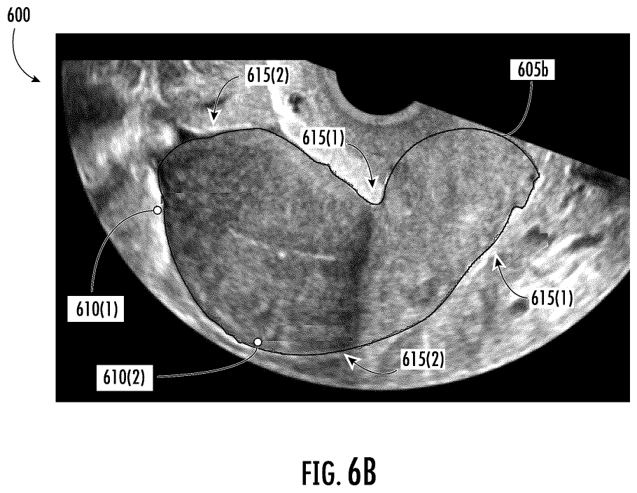

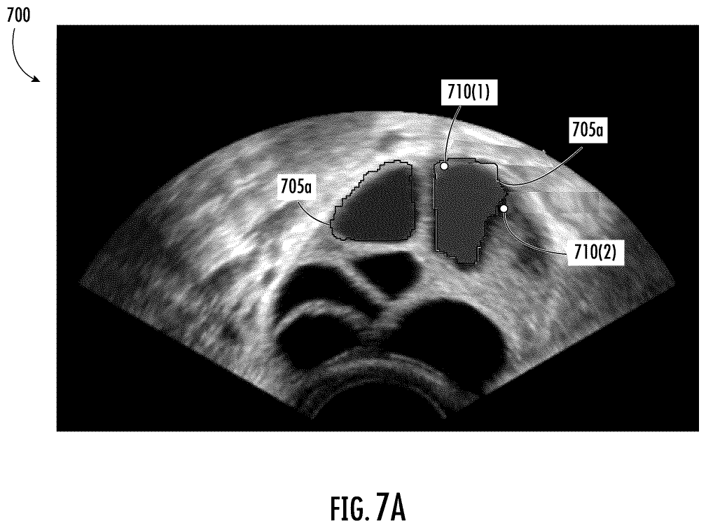

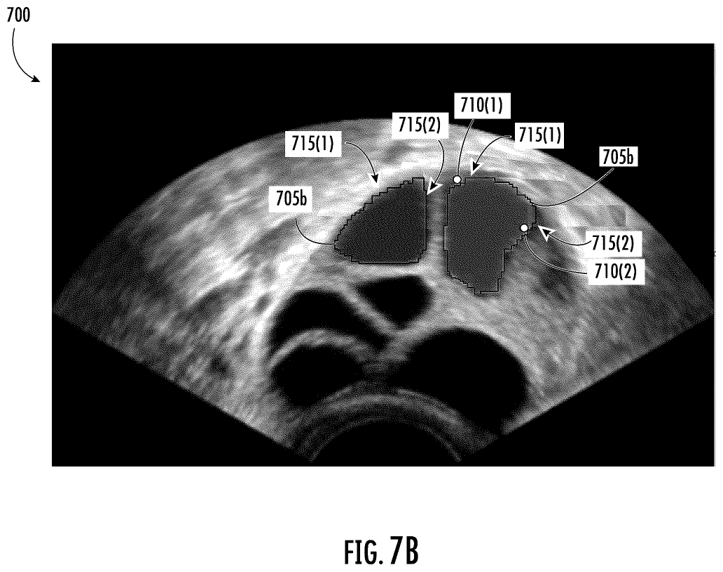

The processor 116 may also be in electronic communication with the display device 132 such that the processor 116 may process ultrasound data obtained by the probe 106 and generate images to display on the display device 132 (e.g., ultrasound image 600 and ultrasound image 700, as described below with reference to FIGS. 6A-6B and 7A-7B, respectively).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120 and the segmentation circuit 122. Both the image processing circuit 120 and the segmentation circuit 122 are configured to facilitate providing user-specific adaptive segmentation of anatomical features in ultrasound images, as described herein.

The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a fetal ultrasound and may therefore include various images of a patient's uterus and the fetal anatomy contained therein. The image processing circuit 120 may include multiple deep learning-based models configured to analyze the image data. For example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

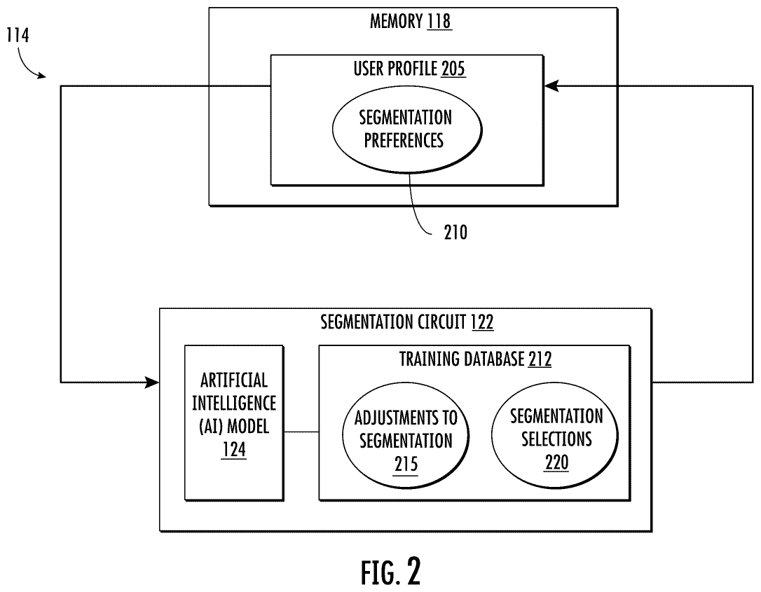

Based on the image analysis performed by the image processing circuit 120, the segmentation circuit 122 is configured to segment anatomical structures identified in the image data. In some embodiments, as shown in FIG. 1, the segmentation circuit 122 may include an artificial intelligence (AI) model 124 configured to perform the segmentation of the anatomical structures, as described below with reference to FIG. 2.

The ultrasound imaging system 100 may also include an external database 128 and a user interface 130. The external database 128 refers to a database from which the processing circuit 114 (e.g., the segmentation circuit 122) may retrieve information used to segment anatomical structures depicted in ultrasound image data. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the processing circuit 114 may retrieve clinical guidelines, standard practices, medical literature, medical textbooks, published research, and previous case studies related to the implementation and/or procedure. For example, if the ultrasound imaging system 100 is being used in a hospital setting to perform fibroid and follicle monitoring, the processing circuit 114 may retrieve clinical guidelines and standard practices related to the hospital setting and the fibroid and follicle monitoring. Continuing with this example, the processing circuit 114 may also retrieve information from the medical literature, medical textbooks, published research, and previous case studies related to uterine and ovarian anatomy.

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

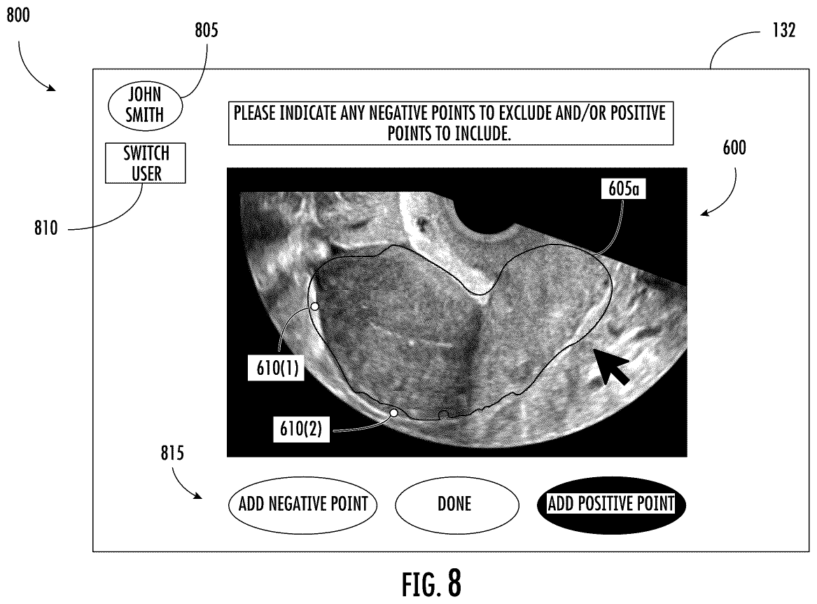

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons. As described below, FIG. 8 illustrates a GUI 800 where at least some of the physical controls of the user interface 130 are presented by various user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring now to FIG. 2, the processing circuit 114 of the ultrasound imaging system 100 is shown in greater detail. More specifically, FIG. 2 depicts the memory 118 and the segmentation circuit 122 configured to facilitate the user-specific adaptive segmentation of anatomical structures in an ultrasound image, as described herein.

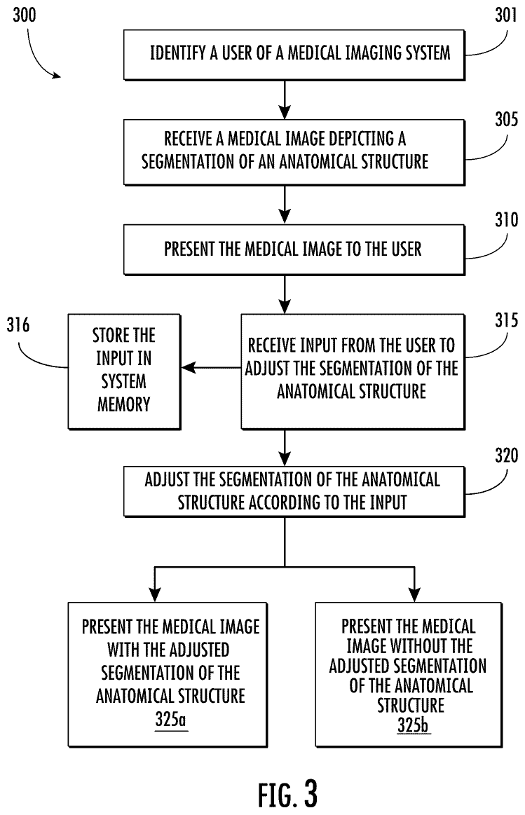

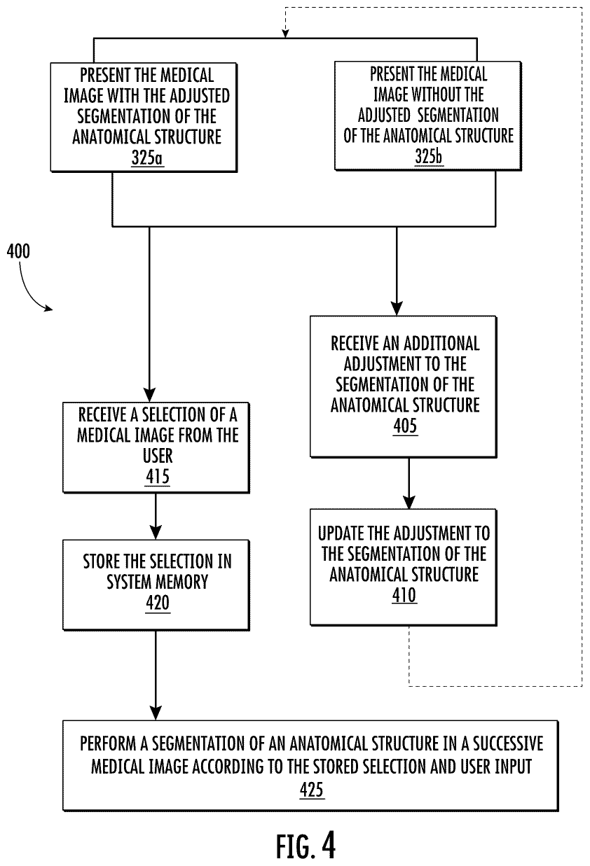

The memory 118 is shown to include a user profile 205. The user profile 205 refers to a profile of an operator (e.g., a sonographer, technician, clinician, etc.) of the ultrasound imaging system 100. For instance, as described below with reference to step 301 of method 300, the ultrasound imaging system 100 may identify the operator prior to initiating collection of ultrasound data. In such instances, the memory 118 may then be configured to retrieve the user profile 205 associated with the identified operator. As shown in FIG. 2, the user profile 205 may include segmentation preferences 210. The segmentation preferences 210 refer to preferences of the user associated with the user profile 205 regarding segmentation of anatomical structures in a medical image. For example, a first user may prefer over-segmentation of anatomical structures, while a second user may prefer under-segmentation of anatomical structures. As described below with reference to FIGS. 3 and 4, the segmentation preferences 210 may be collected and stored in the memory 118 during a medical imaging procedure based on adjustments (e.g., adjustments to a default segmentation) and/or selections received from the user. In this way, the segmentation preferences 210 may be retrieved during successive ultrasound scans upon identifying the user profile 205 associated with the operator of the ultrasound imaging system 100.

As illustrated in FIG. 2, the segmentation preferences 210 from the user profile 205 may be retrieved from the memory 118 and received by the segmentation circuit 122. In this way, while performing a segmentation of an anatomical structure during a medical imaging procedure, the segmentation circuit 122 may segment the anatomical structure according to the received segmentation preferences 210 associated with the user/operator.

The segmentation circuit 122 is shown to include the AI model 124 and a training database 212. In some embodiments, the AI model 124 refers to a segmentation algorithm configured to perform a segmentation of one or more anatomical structures depicted in an image received from the image processing circuit 120. The AI model 124 may be trained according to information stored in the training database 212, including adjustments to segmentation 215 and/or segmentation selections 220, as shown in FIG. 2. The adjustments to segmentation 215 refer to adjustments from the user to a segmentation generated by the segmentation circuit 122 (e.g., input received at step 315 of method 300, additional adjustments received at step 405 of method 400, etc.) and may be reflected in the segmentation preferences 210 of the user profile 205. For example, the adjustments to segmentation may include negative prompts (e.g., negative points 610(1), 710(1)) in response to an over-segmentation of an anatomical structure generated by the segmentation circuit 122. Alternatively or additionally, the adjustments to segmentation may include positive prompts (e.g., positive points 610(2), 710(2)) in response to an under-segmentation of an anatomical structure generated by the segmentation circuit 122. The segmentation selections 220 refer to selections from the user between medical images with varying segmentations of a same anatomical structure (e.g., a selection between the medical image presented at step 325a of method 300 and the medical image presented at step 325b of method 300). The segmentation selections 220 may be reflected in the segmentation preferences 210 of the user profile 205.

As described below with reference to FIGS. 3 and 4, the adjustments to segmentation 215 and/or the segmentation selections 220 may be received from a user during a live medical imaging procedure (e.g., an ultrasound scan), and therefore may be received by the segmentation circuit 122 and then stored in the memory 118 as the segmentation preferences 210. In this way, the adjustments to segmentation 215 and/or the segmentation selections 220 received during a first ultrasound scan may be stored in the memory 118 and applied to the segmentation circuit 122 during a successive ultrasound scan. That is, the AI model 124 may be trained using additional training data from the training database 212 (e.g., the adjustments to segmentation 215 and/or the segmentation selections 220 received during the first ultrasound scan), which may cause the segmentation circuit 122 to perform a segmentation of the anatomical feature during the successive ultrasound scan that differs from the segmentation of the anatomical feature performed by the segmentation circuit 122 during the first ultrasound scan.

Referring to FIG. 3, a flow chart is shown illustrating a method 300 for providing adaptive segmentation of anatomical features in medical images using a medical imaging system. In at least one embodiment, the medical imaging system referred to by method 300 is the ultrasound imaging system 100 described above with reference to FIGS. 1 and 2, and method 300 may be implemented by the ultrasound imaging system 100. In some embodiments, method 300 may be implemented as executable instructions in a memory of the ultrasound imaging system 100, such as the memory 118 of FIG. 1.

Prior to initiating a collection of ultrasound data, method 300 may begin when an operator (e.g., a sonographer, technician, or other clinician) is identified as a user of the ultrasound imaging system 100 at step 301. In some embodiments, the operator may authenticate themselves as an authorized user of the ultrasound imaging system 100 by logging in to a portal (e.g., an online application accessible via the user interface 130) associated with the environment in which the ultrasound imaging system 100 is being implemented (e.g., a hospital or other healthcare provider). For instance, the operator may log in using a unique identifier (e.g., a username, a password, a biometric scan, a pin code, etc.). Once the operator is identified and successfully authenticated, the ultrasound imaging system 100 may be configured to retrieve a user profile (e.g., user profile 205) associated with the identified operator. In some embodiments, the user profile may include various preferences of the operator regarding collection and processing of ultrasound data by the ultrasound imaging system 100.

As shown in FIG. 3, method 300 may include receiving a medical image depicting a segmentation of an anatomical structure at step 305. In some embodiments, receiving the medical image at step 305 may include generating the medical image. For instance, the medical image may be an ultrasound image (e.g., ultrasound image 600, ultrasound image 700) generated by the image processing circuit 120 based on ultrasound data obtained using the probe 106.

The medical image received at step 305 may further include a segmentation of an anatomical structure in the medical image. In some embodiments, the anatomical structure may be segmented at step 305 by the segmentation circuit 122 using a segmentation algorithm (e.g., the AI model 124). The segmentation performed by the segmentation circuit 122 at step 305 may be referred to as a default segmentation (e.g., default segmentations 605a, 705a). That is, the default segmentation refers to a segmentation of the anatomical structure prior to the segmentation being adjusted by any operator input (e.g., as described at step 315 of method 300). In some embodiments, the medical image received at step 305 may depict a plurality of anatomical structures (e.g., as shown in ultrasound image 700 of FIGS. 7A and 7B). In such instances, each of the plurality of anatomical structures may be segmented by the segmentation circuit 122 at step 305.

At step 310, the medical image received at step 305 is presented to the user (e.g., the user of the medical imaging system identified at step 301). That is, the medical image presented to the user includes the segmentation of the anatomical structure. As described below, the medical image may be presented as ultrasound image 600 and/or ultrasound image 700. In some embodiments, such as those shown in FIGS. 6A-8, the segmentation (e.g., default segmentations 605a, 705a) may be illustrated using an outline of the anatomical feature overlaid on the medical image. In some instances, the medical image is presented via the display device 132.

After presenting the medical image to the user at step 310, method 300 includes receiving an input from the user to adjust the segmentation of the anatomical structure as shown in the medical image at step 315. That is, at step 315, the user adjusts the default segmentation generated by the segmentation circuit 122 and presented with the medical image at step 310. The user may adjust the segmentation using the display device 132 (e.g., using a finger to tap points on a touch screen, using a stylus or other probe to select points on the touch screen, etc.).

The input received at step 315 may include at least one of a negative prompt (e.g., negative point 610(1), 710(1)) or a positive prompt (e.g., positive point 610(2), 710(2)). The negative prompt refers to a prompt provided by the user that designates a point on the medical image to exclude from the segmentation of the anatomical structure (e.g., a point previously included in the default segmentation presented at step 310). The positive prompt refers to a prompt provided by the user that designates a point on the medical image to include in the segmentation of the anatomical structure (e.g., a point previously not included in the default segmentation presented at step 310). In other words, a negative prompt is configured to adjust the segmentation in instances of over-segmentation, while the positive prompt is configured to adjust the segmentation in instances of under-segmentation.

The input received from the user at step 315 may then be stored in a memory of the medical imaging system being used to perform method 300 at step 316. For instance, as described above with reference to FIG. 2, the input may be stored in the memory 118 of the ultrasound imaging system 100. More specifically, the input may be stored in relation to the profile of the user identified at step 301 (e.g., the user profile 205). As shown in FIG. 2, the input may be stored in the user profile 205 among the segmentation preferences 210.

At step 320 of method 300, the segmentation of the anatomical structure (e.g., default segmentations 605a, 705a) is adjusted according to the input received at step 315. Where the input received at step 315 includes a negative prompt, for instance, adjusting the segmentation at step 320 may include excluding a plurality of points along an outline of the anatomical structure (e.g., a plurality of points previously included in the segmentation presented at step 310) according to the negative prompt. That is, the segmentation is adjusted in such instances to compensate for over-segmentation (e.g., depicted by adjustments 615(1), 715(1)) previously generated by the segmentation circuit 122. Alternatively or additionally, where the input received at step 315 includes a positive prompt, adjusting the segmentation at step 320 may include adding/including a plurality of points along an outline of the anatomical structure (e.g., a plurality of points previously excluded from the segmentation presented at step 310) according to the positive prompt. That is, the segmentation is adjusted in such instances to compensate for under-segmentation (e.g., depicted by adjustments 615(2), 715(2)) previously generated by the segmentation circuit 122.

According to certain instances, where the medical image presented at step 305 depicts a plurality of anatomical structures, step 320 may include adjusting a segmentation of the plurality of anatomical structures according to the input. That is, as described in greater detail below with reference to FIGS. 7A and 7B, an input received at step 315 to adjust the segmentation of a first anatomical structure may be applied to the plurality of anatomical structures depicted in the medical image. For example, where the input received at step 315 includes a negative prompt (e.g., negative point 710(1)), adjusting the segmentation at step 320 may include excluding a plurality of points along an outline of each of the plurality of anatomical structures according to the negative prompt. Similarly, as another example, where the input received at step 315 includes a positive prompt (e.g., positive point 710(2)), adjusting the segmentation at step 320 may include adding/including a plurality of points along an outline of each of the plurality of anatomical structures according to the positive prompt.

Method 300 continues by presenting the medical image with the adjusted segmentation of the anatomical structure at step 325a. The medical image may be presented at step 325a via the display device 132. In some instances, where the anatomical structure is a first anatomical structure of a plurality of anatomical structures depicted by the medical image, the medical image is presented with the adjusted segmentation of the plurality of anatomical structures (e.g., as depicted by ultrasound image 700 in FIG. 7B).

In some instances, the medical image without the adjusted segmentation of the anatomical structure may be presented at step 325b. According to such instances, steps 325a and 325b may be performed concurrently such that the medical image with the adjusted segmentation of the anatomical structure may be presented alongside (e.g., via a split-screen, etc.) the medical image without the adjusted segmentation (e.g., an unadjusted segmentation) of the anatomical structure. The adjusted segmentation and the unadjusted segmentation may be presented to the user of the ultrasound imaging system via the display device 132. For instance, where the display device 132 includes a touchscreen, the user may engage with the presentation of the adjusted segmentation and/or the presentation of the unadjusted segmentation.

Referring to FIG. 4, a flow chart is shown illustrating a method 400 by which the user may engage with the presentation of the adjusted segmentation (e.g., from step 325a of method 300) and/or the presentation of the unadjusted segmentation (e.g., from step 325b of method 300). In at least one embodiment, the medical imaging system referred to by method 400 is the ultrasound imaging system 100 described above with reference to FIGS. 1 and 2, and method 400 may be implemented by the ultrasound imaging system 100. In some embodiments, method 400 may be implemented as executable instructions in a memory of the ultrasound imaging system 100, such as the memory 118 of FIG. 1.

For instance, upon presenting the medical image with the adjusted segmentation of the anatomical structure at step 325a, the ultrasound imaging system 100 may receive an additional adjustment to the segmentation of the anatomical structure from the user at step 405 of method 400. That is, the user may apply one or more additional negative prompts (e.g., negative points 610(1), 710(1)) to indicate additional points along the outline of the anatomical structure to exclude from the segmentation presented with the medical image at step 325a. Alternatively or additionally, at step 405, the user may apply one or more additional positive prompts (e.g., positive points 610(2), 710(2)) to indicate additional points along the outline of the anatomical structure to include in the segmentation presented with the medical image at step 325a.

Therefore, upon receiving the additional adjustment to the segmentation of the anatomical structure at step 405, method 400 may continue with updating the adjustment to the segmentation of the anatomical structure at step 410. Step 410 may be performed as described above with reference to step 320 of method 300. That is, where the additional adjustment received at step 405 includes a negative prompt, updating the adjustment to the segmentation at step 410 may include excluding a plurality of points along the outline of the anatomical structure (e.g., a plurality of points previously included in the segmentation presented at step 325a) according to the negative prompt. Alternatively or additionally, where the additional adjustment received at step 405 includes a positive prompt, updating the adjustment to the segmentation at step 410 may include adding/including a plurality of points along the outline of the anatomical structure (e.g., a plurality of points previously excluded from the segmentation presented at step 325a) according to the positive prompt.

As shown in FIG. 4, after receiving the update to the adjustment to the segmentation at step 410, the ultrasound imaging system 100 is configured to present the medical image with the adjusted segmentation (e.g., step 325a of method 300) and to present the medical image without the adjusted segmentation (e.g., step 325b). In such instances, however, the adjusted segmentation refers to the updates to the segmentation made at step 410 based on the additional adjustment received at step 405, rather than to the adjustments to the segmentation made at step 320 of method 300 based on the input received from the user at step 315. In other words, FIG. 4 represents an iterative process by which the ultrasound imaging system 100 receives additional adjustments to the segmentation of the anatomical structure from the user (e.g., received at step 405) and presents an updated version of the medical image to reflect such adjustments to the segmentation (e.g., updated at step 410).

In some instances, such as those in which the user has no additional adjustments to make to the segmentation presented at step 325a, the method 400 may continue by receiving a selection of a medical image from the user at step 415. More specifically, the selection refers to a selection between the medical image with the adjusted segmentation presented at step 325a and the medical image without the adjusted segmentation presented at step 325b. For example, if the user approves of the adjusted segmentation of the anatomical structure presented at step 325a, the user may select the medical image with the adjusted segmentation. On the other hand, if the user does not approve of the adjusted segmentation and prefers the version of the medical image without the adjusted segmentation, the user may select the medical image without the adjusted segmentation. For instance, if the user has applied one or more additional adjustments (e.g., at step 405) to the segmentation, the user may prefer the segmentation prior to being updated according to the additional adjustments (e.g., at step 410), and therefore may select the medical image without the adjusted segmentation.

At step 420 of method 400, the selection received at step 415 is stored in a system memory. More specifically, the selection may be stored in the memory 118 as a user preference (e.g., among the segmentation preferences 210) associated with the profile of the user (e.g., the user profile 205) identified at step 301 of method 300.

Therefore, adjustments to the segmentation received from the user (e.g., at step 315, at step 405) and the selection of the medical image are stored by the ultrasound imaging system 100 such that a user preference (e.g., the segmentation preferences 210) associated with the adjustments to the segmentation and/or the selection is applied by the segmentation circuit 122 (e.g., the AI model 124) during a segmentation of the anatomical structure in a successive medical image. In this way, method 400 is shown to include performing a segmentation of an anatomical structure in a success medical image according to the stored selection (e.g., received at step 415) and user input (e.g., received at step 315 of method 300 and at step 405 of method 400).

For example, if the user selects the medical image with the adjusted segmentation of the anatomical structure (e.g., presented at step 325a) at step 415, and that selection is stored in the memory 118 at step 420, step 425 may begin by the ultrasound imaging system 100 (e.g., the image processing circuit 120) identifying the anatomical structure in a successive ultrasound image. Then, based on the identification of the anatomical structure, step 425 may include segmenting, using the segmentation circuit 122, the anatomical structure in the successive ultrasound image based on the input and the selection such that after segmenting the anatomical structure in the successive ultrasound image, the successive ultrasound image resembles the ultrasound image with the adjusted segmentation of the anatomical structure. That is, the segmentation circuit 122 is configured to identify segmentation preferences 210 from the user profile 205 and apply such segmentation preferences 210 to the ultrasound image as the segmentation preferences 210 relate to the anatomical structure identified by the image processing circuit 120.

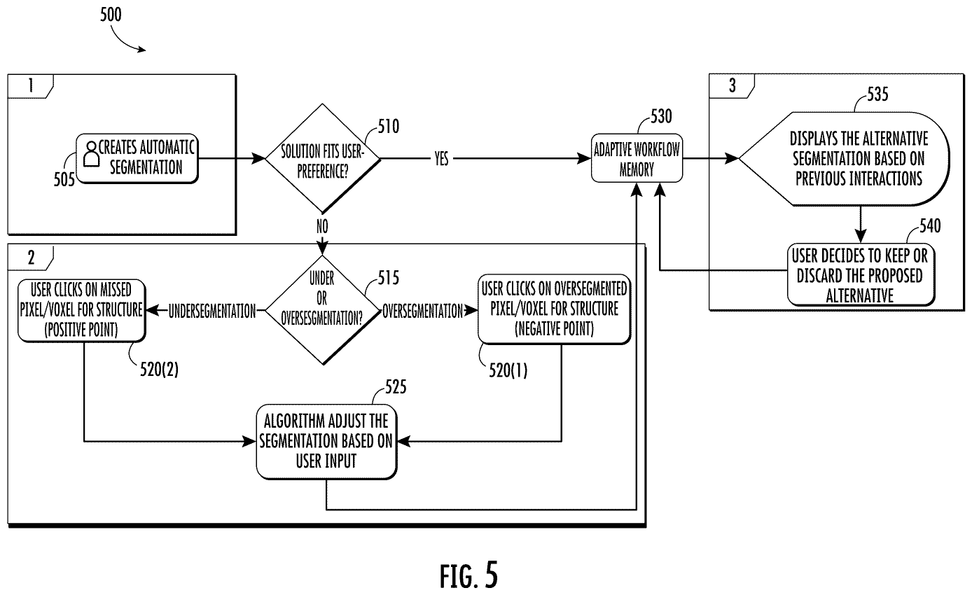

Referring to FIG. 5, a diagram illustrating a workflow 500 for performing adaptive and intelligent segmentation of an anatomical structure is shown. In at least one embodiment, the workflow 500 may be implemented by the ultrasound imaging system 100. In some embodiments, the workflow 500 may be implemented as executable instructions in a memory of the ultrasound imaging system 100, such as the memory 118 of FIG. 1.

The workflow 500 represents a continuous adaptive system that begins with a user initiating an automated segmentation of a desired anatomical structure (e.g., a pre-existing automation algorithm, such as the AI model 124 of the segmentation circuit 122) at step 505 of block 1, as shown in FIG. 5.

Following the intimation of the automatic segmentation at step 505, the workflow 500 includes determining whether the solution (e.g., the automatic segmentation generated in response to the initiation from the user at step 505) fits the preferences of the user (e.g., the segmentation preferences 210).

Where the solution does not fit the user preferences, the workflow 500 proceeds with adjusting the segmentation output at block 2, as shown in FIG. 5. As depicted by step 515 of the workflow 500, the user may determine whether the segmentation output depicts an under-segmentation (e.g., the segmentation excluding points/regions along the outline of the anatomical structure that the user prefers to include in the segmentation) and/or an over-segmentation (e.g., the segmentation including points/regions along the outline of the anatomical structure that the user prefers not to include in the segmentation). In some embodiments, the segmentation output may include only areas of under-segmentation, only areas of over-segmentation, or both areas of under-segmentation and areas of over-segmentation.

The segmentation output may be adjusted as described above with reference to method 300 and/or method 400. That is, the user may adjust the segmentation output with simple and minimal interaction by at least one of marking a point to be excluded on an over-segmentation (e.g., negative prompt) or marking a point to be included because of under-segmentation (e.g., positive prompt). For instance, if the segmentation is determined at step 515 to depict an over-segmentation, the workflow 500 may include the user clicking on over-segmented pixels/voxels (e.g., negative points 610(1), 710(1)) at step 520(1) in order to adjust the structure of the segmentation. Alternatively or additionally, if the segmentation is determined at step 515 to depict an under-segmentation, the workflow 500 may include the user clicking on missed (e.g., excluded, omitted, etc.) pixels/voxels (e.g., positive points 610(2), 710(2)) at step 520(2) in order to adjust the structure of the segmentation.

In response to negative points received at step 520(1) and/or positive points received at step 520(2), the segmentation algorithm (e.g., the segmentation circuit 122) may be configured to adjust the segmentation accordingly at step 525. The prompt received at step 520(1) and/or 520(2) may be used to adjust or alter the entire segmentation using the properties of point (e.g., pixel, voxel, etc.) marked by the user. For example, the segmentation algorithm (e.g., the segmentation circuit 122) may use pixel/voxel properties such as neighborhood histogram distribution, contrast variation, homogeneity of neighborhood pixels, as well as shape smoothness when adjusting the segmentation at step 525. Furthermore, where the medical image includes a plurality (e.g., more than one) of anatomical structures, the adjustments may be propagated using the pixel/voxel properties to multiple instances of a same structure. For example, an ultrasound image obtained during an ovarian ultrasound may depict multiple follicles, and adjustments to a first follicle based on input from the user (e.g., positive points and/or negative points) applied to the first of the multiple follicles may be made to a remainder of the multiple follicles by the segmentation circuit 122.

After adjusting the segmentation, the input may be stored in the system memory (e.g., an adaptive workflow memory) at step 530. As shown in FIG. 5, the adaptive workflow memory allows the workflow 500 to perform a feedback loop (e.g., depicted by block 3) that includes learning user behavior and preference (e.g., based on the segmentations and adjustments saved to the adaptive workflow memory) over time such that the segmentation algorithm is updated according to the learned user behavior and preference. In this way, subsequent segmentation suggestions provided to the user by the segmentation circuit 122 (e.g., provided in response to initiating the automatic segmentation at step 505) require few to no adjustments/edits from the user because the automatically generated segmentation matches the user's preferences and prior behavior.

As shown by block 3 of FIG. 5, the feedback loop of the workflow 500 may include displaying the alternative segmentation at step 535 based on the received user input (e.g., the user's previous interactions with a segmentation generated by the segmentation circuit 122). For instance, where the user applies at least one of a negative point at step 520(1) or a positive point at step 520(2), the alternative segmentation may refer to the segmentation adjusted by the segmentation circuit 122 at step 525. In other words, step 535 may refer to the presentation of the medical image at step 325a of method 300. In this way, the user may also receive a display of the segmentation without the adjustments at step 535 (e.g., as described above with reference to step 325b of method 300). Therefore, the user may be presented with the existing segmentation (e.g., from step 505) and the updated segmentation (e.g., from step 525) such that the user can choose a segmentation from among the existing segmentation (e.g., presented at step 325b) and the updated segmentation (e.g., presented at step 325a) at step 540. In other words, the user decides to keep or discard the proposed alternative segmentation. The feedback loop further includes storing the selection within the adaptive workflow memory (e.g., step 530) such that the default segmentation setting for the user is updated based on the user selection.

Where the solution generated in response the automatic segmentation initiated at step 505 does fit the user preferences, as determined at step 510, the workflow 500 proceeds with storing the segmentation output in the adaptive workflow memory at step 530 and initiating the feedback loop depicted by block 3.

Referring to FIGS. 6A and 6B, an ultrasound image 600 is shown. The ultrasound image 600 may be obtained by the ultrasound imaging system 100 during a uterus examination, and may depict an anatomical structure (e.g., the uterus). In some embodiments, the ultrasound image 600 may be the medical image referred to in method 300 and/or method 400, as described above. Furthermore, the ultrasound image 600 may be provided to a user of the ultrasound imaging system 100 via the display device 132.

As shown in FIG. 6A, the anatomical structure is outlined/segmented according to a default segmentation 605a. That is, the default segmentation 605a may be generated by the segmentation circuit 122 using a pre-existing segmentation algorithm. Alternatively or additionally, the default segmentation 605a may be generated by the segmentation circuit 122 using the segmentation preferences 210 stored among the user profile 205 associated with the user performing the uterus examination. In some embodiments, the ultrasound image 600 with the default segmentation 605a is the medical image received at step 305 and presented to the user at step 310 of method 300.

The ultrasound image 600 is also shown to include a negative point 610(1) and a positive point 610(2). As described above, the negative point 610(1) designates a point on the ultrasound image 600 to exclude from the default segmentation 605a. The positive point 610(2) designates a point on the ultrasound image 600 to include in the default segmentation 605a. In other words, the negative point 610(1) is configured to adjust the default segmentation 605a in instances of over-segmentation, while the positive point 610(2) is configured to adjust the default segmentation 605a in instances of under-segmentation. In some embodiments, the negative point 610(1) and the positive point 610(2) are the input received at step 315 of method 300. That is, the user may designate the negative point 610(1) and the positive point 610(2) by clicking (e.g., using a mouse, a finger, a stylus, a probe, etc.) on the respective points of the ultrasound image 600 presented on a touch screen (e.g., the display device 132).

As shown in FIG. 6B, the segmentation circuit 122 may be configured to adjust the default segmentation 605a shown in FIG. 6A based on the negative point 610(1) and the positive point 610(2), thus resulting in adjusted segmentation 605b. More specifically, the adjusted segmentation 605b depicts areas of retraction 615(1) based on the negative point 610(1) and areas of extension 615(2) based on the positive point 610(2). The areas of retraction 615(1) refer to portions of the default segmentation 605a that are removed from the adjusted segmentation 605b by the segmentation circuit 122 based on the negative point 610(1). In this way, the areas of retraction 615(1) shown along the adjusted segmentation 605b are configured to adjust areas of over-segmentation previously shown in the default segmentation 605a. In a similar way, the areas of extension 615(2) refer to portions of the default segmentation 605a that are added to the adjusted segmentation 605b by the segmentation circuit 122 based on the positive point 610(2). In this way, the areas of extension 615(2) shown along the adjusted segmentation 605b are configured to adjust areas of under-segmentation previously shown in the default segmentation 605a.

As shown in FIG. 6B, the areas of retraction 615(1) and the areas of extension 615(2) are not limited to the areas surround the negative points 610(1) and the positive points 610(2), respectively. Rather, the segmentation circuit 122 is configured to adjust an entirety of the default segmentation 605a according to the negative points 610(1) and the positive points 610(2) using the pixels/voxels at each of the negative points 610(1) and the positive points 610(2) in relation to the pixels/voxels of the entirety of the default segmentation 605a.

Referring to FIGS. 7A and 7B, an ultrasound image 700 is shown. The ultrasound image 700 may be obtained by the ultrasound imaging system 100 during a uterus examination, and may depict a plurality of anatomical structures (e.g., multiple follicles). In some embodiments, the ultrasound image 700 may be the medical image referred to in method 300 and/or method 400, as described above. Furthermore, the ultrasound image 700 may be provided to a user of the ultrasound imaging system 100 via the display device 132.

As shown in FIG. 7A, two of the plurality of anatomical structures are outlined/segmented according to a default segmentation 705a. It should be appreciated that although only two of the plurality of anatomical structures are shown to be outlined according to the default segmentation 705a in FIGS. 7A and 7B, any number of the plurality of anatomical structures may be outlined according to the default segmentation 705a. The default segmentation 705a may be generated by the segmentation circuit 122 using a pre-existing segmentation algorithm. Alternatively or additionally, the default segmentation 705a may be generated by the segmentation circuit 122 using the segmentation preferences 210 stored among the user profile 205 associated with the user performing the uterus examination. In some embodiments, the ultrasound image 700 with the default segmentation 705a is the medical image received at step 305 and presented to the user at step 310 of method 300.

The ultrasound image 700 is also shown to include a negative point 710(1) and a positive point 710(2). As described above, the negative point 710(1) designates a point on the ultrasound image 700 to exclude from the default segmentation 705a. The positive point 710(2) designates a point on the ultrasound image 700 to include in the default segmentation 705a. In other words, the negative point 710(1) is configured to adjust the default segmentation 705a in instances of over-segmentation, while the positive point 710(2) is configured to adjust the default segmentation 705a in instances of under-segmentation. In some embodiments, the negative point 710(1) and the positive point 710(2) are the input received at step 315 of method 300. That is, the user may designate the negative point 710(1) and the positive point 710(2) by clicking (e.g., using a mouse, a finger, a stylus, a probe, etc.) on the respective points of the ultrasound image 700 presented on a touch screen (e.g., the display device 132). As shown in FIG. 7A, the negative point 710(1) and the positive point 710(2) may be designated on only one of the two anatomical structures outlined by the default segmentation 705a.

As shown in FIG. 7B, the segmentation circuit 122 may be configured to adjust the default segmentation 705a shown in FIG. 7A based on the negative point 710(1) and the positive point 710(2), thus resulting in adjusted segmentation 705b. Although the negative point 710(1) and the positive point 710(2) are designated on only one of the two anatomical structures outlined by the default segmentation 705a, the adjusted segmentation 705b may be applied to the two anatomical structures outlined by the default segmentation 705a. That is, the segmentation circuit 122 is configured to apply the adjustments from the user (e.g., the negative point 710(1) and the positive point 710(2)) to any of the plurality of anatomical structures (e.g., to any of the follicles shown in FIGS. 7A and 7B).

More specifically, the adjusted segmentation 705b depicts areas of retraction 715(1) based on the negative point 710(1) and areas of extension 715(2) based on the positive point 710(2). The areas of retraction 715(1) refer to portions of the default segmentation 705a that are removed from the adjusted segmentation 705b by the segmentation circuit 122 based on the negative point 710(1). In this way, the areas of retraction 715(1) shown along the adjusted segmentation 705b are configured to adjust areas of over-segmentation previously shown in the default segmentation 705a. In a similar way, the areas of extension 715(2) refer to portions of the default segmentation 705a that are added to the adjusted segmentation 705b by the segmentation circuit 122 based on the positive point 710(2). In this way, the areas of extension 715(2) shown along the adjusted segmentation 705b are configured to adjust areas of under-segmentation previously shown in the default segmentation 705a.

As shown in FIG. 7B, the areas of retraction 715(1) and the areas of extension 715(2) are not limited to the areas surrounding the negative points 710(1) and the positive points 710(2), respectively. Rather, the segmentation circuit 122 is configured to adjust an entirety of the default segmentation 705a according to the negative points 710(1) and the positive points 710(2) using the pixels/voxels at each of the negative points 710(1) and the positive points 710(2) in relation to the pixels/voxels of the entirety of the default segmentation 705a.

Referring to FIG. 8, a GUI 800 with selectable elements configured to enable a user (e.g., an operator of the ultrasound imaging system 100) to control operation of the ultrasound imaging system 100 by selection thereof is shown. In some embodiments the GUI 800 may be a GUI generated for display on the display device 132. Further, the GUI 800 may be configured as a touch screen display, such that the user may select one of the selectable elements by touching the respective location of the selectable element on the touch screen display. Alternatively or additionally, each of the selectable elements shown on the GUI 800 may be configured as hardware elements (e.g., physical buttons) included as part of the user interface 130.

As shown, the GUI 800 includes an indication of a user profile 805 and an option to switch user 810. The indication of the user profile 805 may be depicted as a name of the user of the ultrasound imaging system 100 (e.g., the user identified at step 301 of method 300). Furthermore, the indication of the user profile 805 may represent the user profile 205 retrieved from the memory 118, as described above. In this way, the GUI 800 may be configured to reflect user preferences (e.g., segmentation preferences 210, etc.) associated with the user profile 205. The option to switch user 810 allows a user of the ultrasound imaging system 100 to switch to a user profile (e.g., user profile 205) other than the user profile currently represented by the indication of the user profile 805.

The GUI is also shown to include the ultrasound image 600 depicting the default segmentation 605a, the negative point 610(1), and the positive point 610(2), as described above with reference to FIG. 6A. Therefore, the GUI 800 may be configured to allow the user of the ultrasound imaging system 100 to adjust the default segmentation 605a by applying the negative point 610(1) and the positive point 610(2) via the GUI 800. For instance, the GUI 800 includes an instruction to “please indicate any negative points to exclude and/or positive points to include” regarding the ultrasound image 600.

As shown in FIG. 8, selectable elements 815 may enable the user to adjust the default segmentation 605a by at least one of adding a negative point (e.g., negative point 610(1)) or adding a positive point (e.g., positive point 610(2)). For example, upon selecting “add positive point” from among the selectable elements 815 displayed via the GUI 800, any point on the ultrasound image 600 thereafter selected by the user (e.g., clicked on, tapped, etc.) may be registered by the segmentation circuit 122 as a point to include in an adjusted segmentation (e.g., the adjusted segmentation 605b) of the uterus depicted in ultrasound image 600. Alternatively or additionally, upon selecting “add negative point” from among the selectable elements 815 displayed via the GUI 800, any point on the ultrasound image 600 thereafter selected by the user (e.g., clicked on, tapped, etc.) may be registered by the segmentation circuit 122 as a point to exclude in an adjusted segmentation (e.g., the adjusted segmentation 605b) of the uterus depicted in ultrasound image 600. After the user has designated the desired positive points and negative points, the user may select “done” from the selectable elements 815, which may prompt the segmentation circuit 122 to adjust the default segmentation 605a based on the negative point 610(1) and the positive point 610(2), and therefore generate the adjusted segmentation 605b. In this way, upon receiving an indication that the user has select “done” from among the selectable elements 815, the GUI 800 may update such that the ultrasound image 600 is depicted on the GUI 800 with the adjusted segmentation 605b including the areas of retraction 615(1) and the areas of extension 615(2), as shown in FIG. 6B.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a transducer configured to transmit and receive an ultrasound signal;
a matching layer configured to have an acoustic impedance between a tissue to be imaged and a material of the transducer;
a damping block configured to absorb ultrasound energy; and
a processing circuit comprising a processor coupled to a memory device storing instructions thereon that, when executed, cause the processing circuit to perform operations comprising:
generating an ultrasound image based on image data obtained by the transducer;
segmenting an anatomical structure in the ultrasound image using a segmentation algorithm;
presenting, on a display of the ultrasound imaging system, the ultrasound image including the segmentation of the anatomical structure;
receiving, via the display, an input from a user, wherein the input comprises an adjustment to the segmentation of the anatomical structure;
adjusting the segmentation of the anatomical structure based on the input;
presenting, via the display, the ultrasound image with the adjusted segmentation of the anatomical structure; and
updating the segmentation algorithm based on the input.

2. The ultrasound imaging system of claim 1, wherein the input comprises at least one of a negative prompt or a positive prompt, wherein the negative prompt designates a point to exclude from the segmentation of the anatomical structure and wherein the positive prompt designates a point to include in the segmentation of the anatomical structure.

3. The ultrasound imaging system of claim 2, wherein the input comprises the negative prompt and wherein adjusting the segmentation of the anatomical structure comprises excluding a plurality of points along an outline of the anatomical structure based on the negative prompt.

4. The ultrasound imaging system of claim 2, wherein the input comprises the positive prompt and wherein adjusting the segmentation of the anatomical structure comprises including a plurality of points along an outline of the anatomical structure based on the positive prompt.

5. The ultrasound imaging system of claim 1, wherein the anatomical structure is a first anatomical structure of a plurality of anatomical structures depicted by the ultrasound image, and wherein the operations further comprise:
adjusting a segmentation of the plurality of anatomical structures according to the input; and
presenting, to the user via the display, the ultrasound image with the adjusted segmentation of the plurality of anatomical structures.

6. The ultrasound imaging system of claim 1, wherein the operations further comprise presenting, via the display, the ultrasound image without the adjusted segmentation of the anatomical structure.

7. The ultrasound imaging system of claim 6, wherein the operations further comprise:
receiving, from the user via the display, a selection of one of the ultrasound image without the adjusted segmentation of the anatomical structure or the ultrasound image with the adjusted segmentation of the anatomical structure; and
storing the input from the user and the selection from the user such that a user preference associated with the input and the selection is applied by the segmentation algorithm during a segmentation of the anatomical structure in a successive ultrasound image.

8. The ultrasound imaging system of claim 7, wherein the user selects the ultrasound image with the adjusted segmentation of the anatomical structure and wherein the operations further comprise:
identifying the anatomical structure in the successive ultrasound image; and
segmenting, using the segmentation algorithm, the anatomical structure in the successive ultrasound image based on the input and the selection such that after segmenting the anatomical structure in the successive ultrasound image, the successive ultrasound image resembles the ultrasound image with the adjusted segmentation of the anatomical structure.

9. The ultrasound imaging system of claim 1, wherein the segmentation algorithm comprises a machine learning model, and wherein the input from the user is stored in a training database used to train the machine learning model.

10. A medical imaging system comprising:
a processing circuit having a processor coupled to a memory device storing instructions thereon that, when executed, cause the processing circuit to perform operations comprising:
generating a medical image;
segmenting an anatomical structure in the medical image using a segmentation algorithm;
presenting, on a display of the medical imaging system, the medical image including the segmentation of the anatomical structure;
receiving, via the display, an input from a user, wherein the input comprises an adjustment to the segmentation of the anatomical structure;
adjusting the segmentation of the anatomical structure based on the input;
presenting, via the display, the medical image with the adjusted segmentation of the anatomical structure; and
updating the segmentation algorithm based on the input from the user.

11. The medical imaging system of claim 10, wherein the medical imaging system comprises an ultrasound imaging system comprising:
a transducer configured to transmit and receive an ultrasound signal;
a matching layer configured to have an acoustic impedance between a tissue to be imaged and a material of the transducer; and
a damping block configured to absorb ultrasound energy.

12. The medical imaging system of claim 10, wherein the anatomical structure is depicted as a two-dimensional structure or a three-dimensional structure in the medical image.

13. The medical imaging system of claim 10, wherein the input comprises at least one of a negative prompt or a positive prompt, wherein the negative prompt designates a point to exclude from the segmentation of the anatomical structure and wherein the positive prompt designates a point to include in the segmentation of the anatomical structure.

14. The medical imaging system of claim 13, wherein the anatomical structure is a first anatomical structure of a plurality of anatomical structures depicted by the medical image, and wherein the operations further comprise:
adjusting a segmentation of the plurality of anatomical structures according to the input; and
presenting, to the user via the display, the medical image with the adjusted segmentation of the plurality of anatomical structures.

15. The medical imaging system of claim 10, wherein the operations further comprise:
presenting, via the display, the medical image without the adjusted segmentation of the anatomical structure;
receiving, from the user via the display, a selection of one of the medical image without the adjusted segmentation of the anatomical structure or the medical image with the adjusted segmentation of the anatomical structure; and
storing the input from the user and the selection from the user such that a user preference associated with the input and the selection is applied by the segmentation algorithm during a segmentation of the anatomical structure in a successive medical image.

16. A method comprising:
generating, by a processing circuit of a medical imaging system, a medical image;
segmenting, by the processing circuit, an anatomical structure in the medical image using a segmentation algorithm;
presenting, by the processing circuit, the medical image including the segmentation of the anatomical structure;
receiving, by the processing circuit, an input from a user, wherein the input comprises an adjustment to the segmentation of the anatomical structure;
adjusting, by the processing circuit, the segmentation of the anatomical structure based on the input;
presenting, by the processing circuit, the medical image with the adjusted segmentation of the anatomical structure; and
updating the segmentation algorithm based on the input from the user.

17. The method of claim 16, wherein the medical imaging system is an ultrasound imaging system, and wherein the medical image is an ultrasound image.

18. The method of claim 16, wherein the input comprises at least one of a negative prompt or a positive prompt, wherein the negative prompt designates a point to exclude from the segmentation of the anatomical structure and wherein the positive prompt designates a point to include in the segmentation of the anatomical structure.

19. The method of claim 16, wherein the anatomical structure is a first anatomical structure of a plurality of anatomical structures depicted by the medical image, and wherein the method further comprises:
adjusting, by the processing circuit, a segmentation of the plurality of anatomical structures according to the input; and
presenting, by the processing circuit and to the user, the medical image with the adjusted segmentation of the plurality of anatomical structures.

20. The method of claim 16, wherein the method further comprises:
presenting, by the processing circuit, the medical image without the adjusted segmentation of the anatomical structure;
receiving, by the processing circuit from the user, a selection of one of the medical image without the adjusted segmentation of the anatomical structure or the medical image with the adjusted segmentation of the anatomical structure; and
storing, by the processing circuit, the input from the user and the selection from the user such that a user preference associated with the input and the selection is applied by the segmentation algorithm during a segmentation of the anatomical structure in a successive medical image.
